(19) United States
(12) Patent Application Publication
(10) Pub. No.: US 2019/0381082 A1
(43) Pub. Date: Dec. 19, 2019

(54) APPLICATION OF VERBASCOSIDE IN TREATMENT AND PREVENTION OF TYPE II DIABETIC KIDNEY DISEASE

(71) Applicant: Nanjing University of Chinese Medicine, Nanjing (CN)

(72) Inventors: Shulan SU, Nanjing (CN); Jinao DUAN, Nanjing (CN); Dawei QIAN, Nanjing (CN); Yue ZHU, Janjing (CN); Hui YAN, Nanjing (CN); Xinxin DAI, Nanjing (CN)

(21) Appl. No.: 16/236,403

(22) Filed: Dec. 29, 2018

(30) Foreign Application Priority Data
Jun. 15, 2018 (CN) 201810619230.4

(51) Int. Cl. A61K 31/7034; A61P 3/10; A61P 13/12

(52) U.S. Cl. CPC A61K 31/7034; A61P 13/12; A61P 3/10

(57) ABSTRACT

Disclosed is an application of verbascoside in the preparation of drugs for preventing and treating type II diabetic nephropathy. The protective effect of verbascoside on type II diabetic nephropathy and the action mechanism thereof are also included in the invention. The results show that verbascoside may improve liver injuries caused by high glucose, and reduce levels of serum creatinine, urea nitrogen, microalbuminuria and blood lipid (total cholesterol and triglyceride), fasting blood sugar and serum insulin in spontaneous diabetic db/db mice, and significantly reduce expressions of TGF-β1 and its signal transduction protein Smad3 and Smad4 and α-SMA in kidney tissues. Meanwhile, verbascoside may improve liver injuries caused by high glucose, and inhibit HK-2 proliferation and EMT formation. In conclusion, verbascoside induces significant protection on type II diabetic nephropathy, and its action mechanism is to protect kidney by regulating oxidative stress response, inhibiting TGF-β/smad signal pathways and improving renal fibrosis.

### CROSS-REFERENCE TO RELATED APPLICATIONS

This application claims the benefit of priority from Chinese Patent Application No. 201810619230.4, filed on Jun. 15, 2018. The content of the aforementioned application, including any intervening amendments thereto, is incorporated herein by reference in its entirety.

### TECHNICAL FIELD

The present invention relates to medical technology, and in particular to an application of verbascoside in the preparation of drugs or healthcare products for preventing or treating a diabetic-associated kidney injury.

### BACKGROUND OF THE PRESENT INVENTION

Verbascoside is a representative component of phenylethanoid glycosides, and is widely distributed in a variety of plant families such as Verbenaceae, Labiatae, Scrophulariaceae, Oleaceae and Plantaginaceae. Generally speaking, verbascoside is formedby caffeic acid and hydroxytyrosol via ester and glycoside bonds, and its glycosyl group consists of rhamnose and glucose. It has been found that the oral bioavailability of verbascoside in rats is low, because it undergoes multiple hydrolysis processes in the gastrointestinal tract before being absorbed into blood, and then further undergoes stage I (redox) and/or stage II (glucuronic acid, sulfuric acid and methylation) metabolisms. As reported in the literatures, verbascoside has wide pharmacological activities, such as kidney protection, antibiosis, antioxidation, anti-hypertension, nerve protection, liver protection and anti-inflammation.

Verbascoside in Rehmannia has many biological activities, such as antioxidation, anti-inflammation and sterilization, and may protect liver and lung tissues and skins, and antagonize nervous system injuries. Therefore, verbascoside in Rehmannia is not only used in human healthcare, but also in animal production. With the gradually deepened research, verbascoside will have a broader prospective in the future.

Total saponins from leaves of Rehmanniaglutinosa (TLR) are phenylethanoid glycoside components extracted from leaves of Rehmannia glutinosa, of which verbascoside is a main component. It is mainly used for the treatment of chronic glomerulonephritis and other kidney diseases. As reported in the literatures, verbascoside has pharmacological effects such as memory enhancement, nerve protection, antioxidation and anti-tumor. Because of strong biological activity, small toxic and side effect and wide source, verbascoside has caught much attention from many researchers at home and abroad. As investigated by Shen Xin, et al, verbascoside has a certain immunoregulatory activity,and as found in previous studies, verbascoside is also capable of improving the renal function injury of nephrotoxic nephritis rats model without causing obvious adverse effects. As reported, verbascoside further induces an effect of invigorating kidney and strengthening yang on kidney-yang deficiency mice model established by intraperitoneal injection of oxycortisone. In addition, verbascoside has been proved to be capable of promoting skin repair and improving skin inflammation through antioxidation, reactive oxygen species removal and induction of glutathione S-transferase (GST) activity.

To sum up, the gradually deepened research on Rehmanniaglutinosa promotes the research on its leaves. Many compounds, especially iridoid glycosides and phenylethanoid glycosides, have been separated out from leaves of Rehmanniaglutinosa, laying a foundation for the rational development and utilization of the resources of Rehmanniaglutinosa leaves. The verbascoside contained in leaves of Rehmanniaglutinosa has wide pharmacological effects, as well as a variety of biological activities and good safety, showing potential development value. At present, there is no report on application of verbascoside in the preparation of drugs or healthcare products for preventing or treating a diabetic-associated kidney injury.

### SUMMARY OF THE PRESENT INVENTION

An object of the present invention is to provide an application of verbascoside in the preparation of drugs or healthcare products for preventing or treating a diabetic kidney injury.

The present invention employs spontaneous diabetic db/db mice as an experimental animal model, and uses TLR to perform an intervention treatment. The improvement of TLR on the early kidney injury caused by diabetes is further investigated and revealed by analyzing biochemical indexes, pathological sections of kidney and analyzing TGF-β/smad signal pathways by a Western blotting method.

In the present invention, human renal tubular epithelial (HK-2) cells are selected to establish a high sugar-induced HK-2 cell injury model, and TLR capsules, TLR extract and its major active components verbascoside and catalpol are used for intervention. The expression quantity of related proteins and mRNA in each cell is detected using Western blotting method and Real-time PCR, and the secretion of related factors in cell supernatant is detected using ELISA. In addition,the effect of TLR on regulating oxidative stress reactions of HK-2 cells and TGF-β/smad signal pathway is further explored.

Summary of the present invention is described as follows.

An application of verbascoside in the preparation of drugs or healthcare products for preventing or treating a diabetic kidney injury.

An application of verbascoside as an only component in the preparation of drugs or healthcare products for preventing or treating a diabetic kidney injury.

An application of verbascoside in the preparation of drugs or healthcare products for reducing TGF-β1/Smad signal pathway and α-SMA protein expression.

An application of verbascoside in the preparation of drugs or healthcare products for reducing protein expression quantity of NOX1, NOX2, NoX4, α-SMA, NF-κB p65, TGF-β1, Smad2, Smad3 and Smad4.

An application of verbascoside in the preparation of drugs or healthcare products for increasing protein expression quantity of E-cadherin and Smad7.

An application of verbascoside in the preparation of drugs or healthcare products for reducing expression quantity of MCP-1, IL-1β, TNF-α and IL-6 in HK-2 cell supernatant.

An application of verbascoside in the preparation of drugs or healthcare products for inhibiting formation of EMT.

An application of verbascoside in the preparation of drugs or healthcare products for reducing intracellular reactive oxygen species (ROS) level.

An application of verbascoside in the preparation of drugs for preventing and treating a diabetic kidney injury and in preparation of drugs and healthcare products for improving a liver injury caused by high glucose.

The diabetic-associated kidney injury diseases include tubulointerstitial fibrosis, glomerular hyperfiltration, renal hypertrophy and forepart glomerular sclerosis.

The drugs or healthcare products are administrated in the routes of oral, injection, mucosal or transdermal administration, or the like.

The drugs or healthcare products include tablets, capsules, granules, oral liquid, patches and gels.

The beneficial effects of the present invention are as follows.

1. As shown in PAS results, compared to the control group, the db/db mice in the model group has a glomerular of larger area, a thicker basement membranes and a significantly greater mean OD value (P<0.05).As shown in HE results, the thickness of a large number of renal cystic epithelium in cortex is increased, and hydropic degeneration, cellular swelling and cytoplasm loosening and understain of many renal tubular epithelial cells together with an extremely small amount of casts are observed at the junction of cortex and medulla. The acidophilia reduction of cytoplasm in renal tubular epithelial cells is observed in a small area of the cortex, and proliferation of a small amount of connective tissues is observed in the mesenchyma. The results of histological examination are consistent with those of biochemical analysis (see FIG. 2), that is, the results of the irbesartan group and the verbascoside group are close to those of the control group.

2. Compared to the blank control group, in HK-2 cells with high glucose-induced injuries, the protein expression levels of NOX1, NOX2, NOX4, α-SMA, NF-κB p65, TGF-β1, Smad2, Smad3 and Smad4 are increased significantly (P<0.05), while the protein expression levels of E-cadherin and Smad7 are decreased significantly (P<0.05). After being intervened with 50 μmol/L of verbascoside, the expressions of NOX1, NOX2, NOX4, α-SMA, E-cadherin, NF-κB p65, TGF-β1, Smad2, Smad3, Smad4 and Smad7 return to normal.

3. Compared to the control group, in HK-2 cells with high glucose-induced injuries, the expressions of NOX1, NOX2, NOX4, α-SMA, E-cadherin, NF-κB p65, TGF-β1, Smad2, Smad3, Smad4 and Smad7 are consistent with their protein expressions. After being intervened with 50 μmol/L of verbascoside, the mRNA expressions of NOX1, NOX2, NOX4, α-SMA, E-cadherin, NF-κB p65, TGF-β1, Smad2, Smad3, Smad4 and Smad7 return to normal.

4. Compared to the control group, the expressions of MCP-1, IL-1β, TNF-α and IL-6 in the supernatant of HK-2 cells with high glucose-induced injuries are increased significantly (P<0.05). After being intervened with 50 μmol/L of verbascoside, the expressions of MCP-1, IL-1β, TNF-α and IL-6 return to normal.

5. As shown in the results of biochemical analysis, compared to the db/db model group, the levels of serum creatinine, urea nitrogen and urine microalbuminuria are significantly improved in the treatment group, and the levels of blood lipid (total cholesterol and triglyceride) and blood sugar (fasting blood glucose and blood insulin) are also improved to different extents in the administration group. In addition, the liver indexes (glutamic-oxalacetic transaminase and glutamic-pyruvic transaminase contents) are increased significantly in the model group, which indicates that high glucose causes the liver injury, and TLR may improve the liver injury caused by high glucose. As shown in Western blotting results, the expressions of TGF-β1 and its signal transduction proteins Smad3 and Smad4 and α-SMA protein in kidney tissues are decreased significantly, especially after administration of TLR extract. In conclusion, TLR may reduce the contents of microalbuminuria, serum creatinine and urea nitrogen, improve fasting blood glucose, insulin level and dyslipidemia, reduce the expressions of TGF-β1/Smad signal pathway and α-SMA protein, and improve renal interstitial fibrosis, thereby playing a role in improving the kidney injury.

### BRIEF DESCRIPTION OF THE DRAWINGS

FIG. 1 shows the results of indexes determination (FBG, T-CHO, TG, GOT, GPT, INS, BUN, mALB, Scr) of mice in control group, model group and administration group; #P<0.05; ##P<0.01; ###P<0.001: model group vs. control group; *P<0.05; **P<0.01; ***P<0.001: administration group vs. model group.

FIG. 2 shows the results of pathological section analysis of kidneys of mice in control, model and administration groups; HE (×400) and PAS (×400) #P<0.05; ###P<0.001: model group vs. control group; *P<0.05; **P<0.01: administration group vs. model group. In HE, acidophilia reduction of cytoplasm of renal tubular epithelial cells and proliferation of a small amount of connective tissues are observed.

FIG. 3 shows protein expression levels of α-SMA, TGF-β1, Smad3, Smad4 in kidney tissues determined using Western blotting method; #P<0.05; ##P<0.01: model group vs. control group; *P<0.05;**P<0.01: administration group vs. model group.

FIG. 4 shows (A) evaluation of QC samples and PCA score plots of QC samples (PC1 vs. PC2); (B) QC sample trend chart displaying change of T in all observations, wherein X-axis number indicates sample number (48 samples), and Y-axis is arbitrary.

FIG. 5 shows total ion flow graphs of mouse serum samples under positive and negative ion modes: A- control group serum under positive ion mode; B- model group serum under positive ion mode; C- control group serum under negative ion mode; and D- model group serum under negative ion mode.

FIG. 6 shows OPLS-DA score plot (A), S-plot (B) and VIP plot (C) of a control group (red) and a model group (black) of mouse serum (A1, A2, B1, B2) samples under positive (A1, B1, C1) and negative (A2, B2, C2) ion modes.

FIG. 7 shows PLS-DA score plots of mouse serum (S1, S2) of a control group, a model group and an administration group under positive (S1) and negative (S2) ion modes.

FIG. 8 shows changes in relative intensity of endogenous metabolites identified using UPLC-QTOF/MS; ##P<0.01: model group vs. control group; *P<0.05;**P<0.01: administration group vs. model group.

FIG. 9 shows HK-2 cell morphology images of different groups.

FIG. 10 shows influence of verbascoside on protein expression levels of NOX1, NOX2, NOX4, α-SMA, E-cadherin, NF-κB p65, TGF-β1, Smad2, Smad3, Smad4 and Smad7 in HK-2 cells (##P<0.01: model group vs. control group; *P<0.05; **P<0.01: administration group vs. model group).

FIG. 11 shows influence of verbascoside on mRNA expression levels of NOX1, NOX2, NOX4, α-SMA, E-cadherin, NF-κB p65, TGF-β1, Smad2, Smad3, Smad4 and Smad7in HK-2 cells (#P<0.05; ##P<0.01; ###P<0.001: model group vs. control group; *P<0.05; **P<0.01; ***P<0.001: administration group vs. model group).

FIG. 12 shows influence of verbascoside on secretions of MCP-1, IL-1β, TNF-α and IL-6 in HK-2 cells (###P<0.001: model group vs. control group; **P<0.01; ***P<0.001: administration group vs. model group).

### DETAILED DESCRIPTION OF THE PRESENT INVENTION

The above-mentioned contents of the present invention will be further described below in detail with reference to the embodiments, but it should not be understood that the scope of the present invention is merely limited to the following embodiments. In addition, all technologies achieved based on the above-mentioned contents of the present invention fall within the scope of the present invention.

Example 1: preparation of tablets with 10 g of verbascoside and excipients according to a conventional method.

Example 2: preparation of capsules with 10 g of verbascoside and excipients according to a conventional method.

Example 3: preparation of oral liquids with 10 g of verbascoside and excipients according to a conventional method.

Example 4: researches on effect of verbascoside on improving early kidney injury in db/db mice and its mechanism.

1. Materials and Reagents

1.1 Experimental Animals

32 male db/db mice and 10 male db/m mice of SPF grade, aged 6-8 weeks and weighing 25-40 g, were purchased from Nanjing Biomedical Researchlnstitute of Nanjing University with an animal license number of SCXK (Su 2015-0001).The mice were raised in an animal room under alternating light and dark every 12 hours with constant temperature of 22±2° C. and humidity of 55±10%.

1.2 Drugs and Reagents

Metformin (Squibb Pharmaceutical Co., Ltd.); irbesartan (Sanofi Winthrop Industrie, France); formic acid, acetonitrile and the like of HPLC grade, purchased from Merck Company; Millipore ultrapure water; and serum urea nitrogen (BUN) kit, glutamic-pyruvic transaminase (GPT) kit, glutamic-oxalacetic transaminase (GOT) kit, total cholesterol (T-CHO) kit, insulin (INS) kit, triglyceride (TG) kit, serum creatinine (Scr) kit and microalbuminuria (mALB) kit, purchased from Nanjing Jiancheng Bioengineering Institute. NADPH oxidase subunits: NOX1, NOX2, NOX4, α-smooth muscle actin (α-SMA), E-cadherin, NF-κB p65, TGF-β1, Smad2, Smad3, Smad4 and Smad7, purchased from Abcam (Cambridge, UK); penicillin/streptomycin solution (Nanjing KeyGEN BioTECH KGY002); 0.25% trypsin-EDTA (Nanjing KeyGEN BioTECH KGY001); PBS (Nanjing KeyGEN BioTECH KGB500); DMEM (GIBCO, America, 12800-082); F12 (GIBCO, America, 883684); FBS (ExCell Biology, America, FBS500); 96-well cell culture plate (Corning Incorporated, America, 3516). Other chemicals and reagents used in this study were analytical grade. Verbascoside (batch number: 16012703, mass fraction: 99.57%), purchased from National Institutes for Food and Drug Control.

1.3 Instruments

ACQUITY™ UPLC ultra-high performance liquid chromatography system, Xevo™ TQ mass spectrometry system and Masslynx 4.1 mass spectrometry workstation software (Waters, America); ML204, MS105 analytical balance (Mettler Toledo Instruments Co., Ltd.); Millipore Direct-Q3 Advantage ultra-pure water system (Millipore Co., Ltd.); WH-1 micro-vortex mixer (Shanghai Huxi Analysis Instrument Factory CO., Ltd); KH-500 ultrasonic cleaner (Kunshan Hechuang Ultrasound Instrument Co., Ltd.); Microfuge 22R Centrifuge (Beckman Coulter, America); MX-S adjustable mixer (DLAB Scientific Co., Ltd.); GA-3 Sinocare blood glucose instrument and blood glucose test paper; Suhua super-clean workbench (SW-CJ-1FD); SANYO CO₂ incubator (XD-101); biological inverted microscope (OLYMPUS IX51); constant-temperature water bath (Changzhou Guohua, HH-4); cell culture flask (FALCON, America, 353014); PCR circulator (Labnet, America) and fluorescent quantitative PCR circulator (Zhongshan DAAN, DA7600).

2. Methods and Results

2.1 Grouping and Administration of Experimental Animals

All mice were adaptively fed for 3 weeks, and db/m mice were used as a control group with a total number of 10. Db/db mice were randomly divided into four groups according to blood sugar and body weight each of 8 mice, and the four groups respectively were a model group (M), a metformin group (EJSG, 250 mg·kg⁻¹·d⁻¹), an irbesartan group (EBST, 50 mg·kg^{31 1}·d⁻¹) and a verbascoside group (MRHTG, 70.8 mg·kg⁻¹·d⁻¹). The same amount of saline was intragastrically administered(ig) to the mice in the control group and the model group, and the volume of intragastric administration (ig) was 10 mL√kg⁻¹, and such administration was performed once a day for continuous 6 weeks.

2.2 Specimen Collection and Index Determination

One day before the end of the experiment, urine was collected in a metabolic cage for 12 hours and the urine volume was recorded. Microalbuminuria content was measured and fasting blood glucose (FBG) of mice in each group was measured by tail vein blood sampling. After drug intervention, mice were anesthetized with intraperitoneal injection of 10% chloral hydrate,and blood was collected from eyeballs. The blood was centrifuged at 3000 rpm and 4° C. for 10 minutes to obtain a serum. Theserum was then subjected to determination of serum creatinine, blood urea nitrogen, blood insulin, cholesterol, triglyceride, glutamic-pyruvic transaminase and glutamic-oxalacetic transaminase contents. Kidneys of the mice were collected, and a left kidney was used for pathological sections, and a right kidney was de-enveloped and cleaned for weighing. Subsequently, the kidney was temporarily placed in liquid nitrogen and then stored at −80° C. for Western blotting analysis.

2.2.1 Results and Analysis of Biochemical Indexes

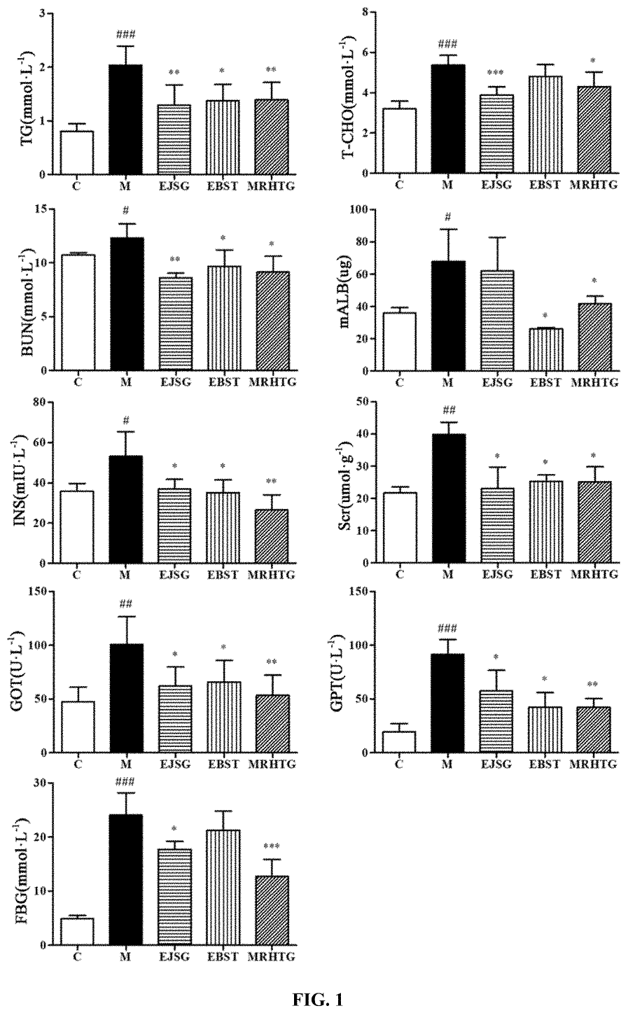

Results of biochemical analysis were shown in FIG. 1. After 6 weeks of administration, compared to the control group, the contents of FBG, T-CHO, TG, GOT, GPT, INS, BUN, mALB and Scr in db/db mice were significantly increased (P<0.05 or P<0.01 or P<0.001). After metformin (250 mg·kg⁻¹·d⁻¹), irbesartan (50 mg·kg⁻¹·d⁻¹) and verbascoside (70.8 mg·kg⁻¹·d⁻¹) were administered, the above indexes all tended to be normal.

2.2.2 Results and Analysis of Pathological Sections

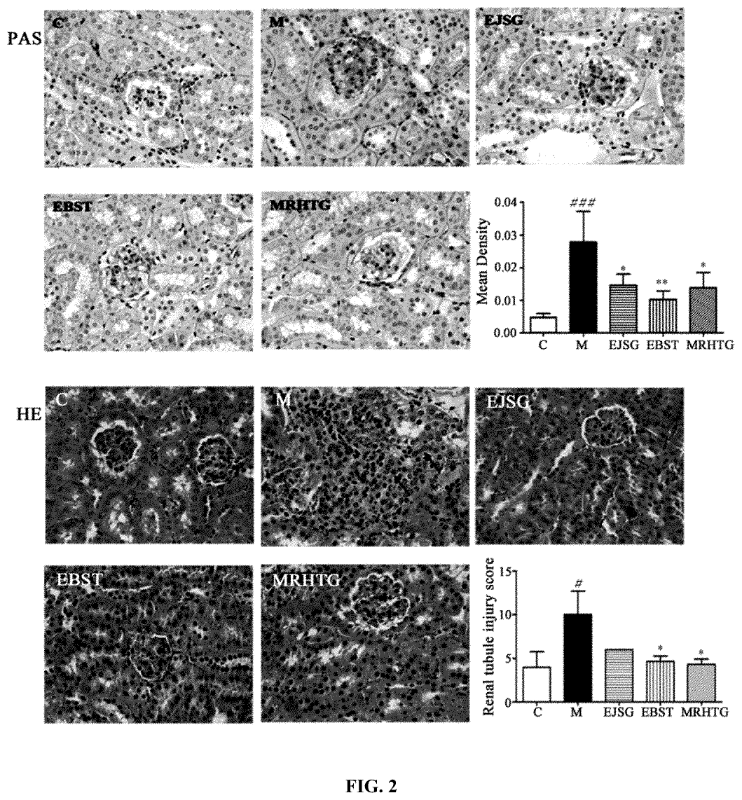

Analysis of PAS mean OD was performed as follows. At least five 400-fold fields of vision of each section were randomly selected for photographing, and kidney tissues were allowed to fill the whole field of vision as far as possible to ensure the consistence among the background light of each photo. A same red-purple color selected using Image-Pro Plus 6.0 software was used as a unified criterion to judgewhetherthe photo was positive. The integrated optical density (IOD) of positive expression of basement membrane and the pixel area (AREA) of glomerular vascular plexus were obtained by analyzing each photo, and the mean OD was calculated as IOD/AREA. As shown in PAS results, compared to the control group, the db/db mice in the model group had a larger glomerular area, a thicker basement membrane and a significantly greater mean OD value (P<0.05). As shown in HE results, the thicknesses of a large number of capsular epithelium in cortex were increased, and hydropic degeneration, cellular swelling and cytoplasm loosening and understain of many renal tubular epithelial cells together with an extremely small amount of casts were observed at the junction of cortex and medulla. The acidophilia reduction of cytoplasm in renal tubular epithelial cells was found in a small area of the cortex, and a small amount of connective tissues proliferation was observed in the mesenchyma. The results of histological examination were consistent with those of biochemical analysis (see FIG. 2), which indicated that the results of the irbesartan group and the verbascoside group were close to those of the control group.

2.2.3 Western Blotting Results and Analysis

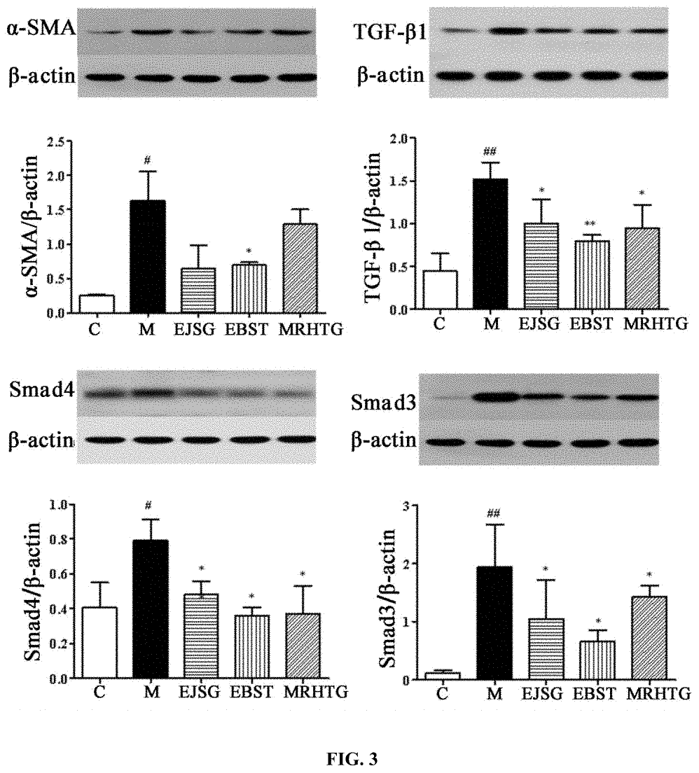

As shown in Western blotting (FIG. 3) results, compared to the control group, the protein expressions of α-SMA, TFG-β1, Smad3 and Smad4 in kidney tissues of db/db mice were increased significantly (P<0.05). In each administration group, the protein expression was returned to that of the control group.

2.3 Method and Results of Metabonomic

2.3.1 Sample Collection and Treatment

After 6 weeks of administration, each of the mice was subjected to blood collection by removing eyeballs. The blood sample was centrifuged at 4° C. and 3000 rpm for 10 minutes to obtain a serum. 100 μL of the frozen-thawed serum was mixed uniformly with 300 μL of acetonitrile under vortex for 2 minutes to produce a mixture. The mixture was centrifuged at 4° C. and 13000 rpm for 10 minutes, and the protein precipitate was removed while the supernatant was collected for determination.

2.3.2 Sample Analysis Parameters

Chromatographic conditions: ACQUITY™ UPLC BEH C18 column (2.1 mm×100 mm, 1.7 μm); and mobile phase: 0.1% formic acid aqueous solution (A)−acetonitrile (B). The gradient elution of serum was programmed as follows: 0-3 minutes, 95%-55% A; 4-13 minutes, 55%-5% A; 13-14 minutes, 5% A. The injection volume was 2 μL and a column temperature of 35° C. was employed.

Mass spectrometry conditions: ESI ion source (ESI⁺/ESI⁻); mass scanning range: 100-1000 m/z;a capillary voltage of 3.0 kV and a cone voltage of 30 V;an extraction voltage of 2.0 V; ion source temperature and desolvation temperature: respectively 120° C. and 350° C.; cone gas flow rate: 50 L/h; collision energy: 20-50 eV; desolvation gas flow rate: 600 L/h; activation time: 30 ms; and collision gas: high-purity nitrogen. Leucine-enkephalin (ESI⁻: 555.2615 m/z, EST⁻: 556.2771 m/z) solution of 200 μg/mL was used as a mass-locking solution at a flow rate of 100 μL/min.

2.3.3 Quality Control (QC) Sample Treatment and Analysis

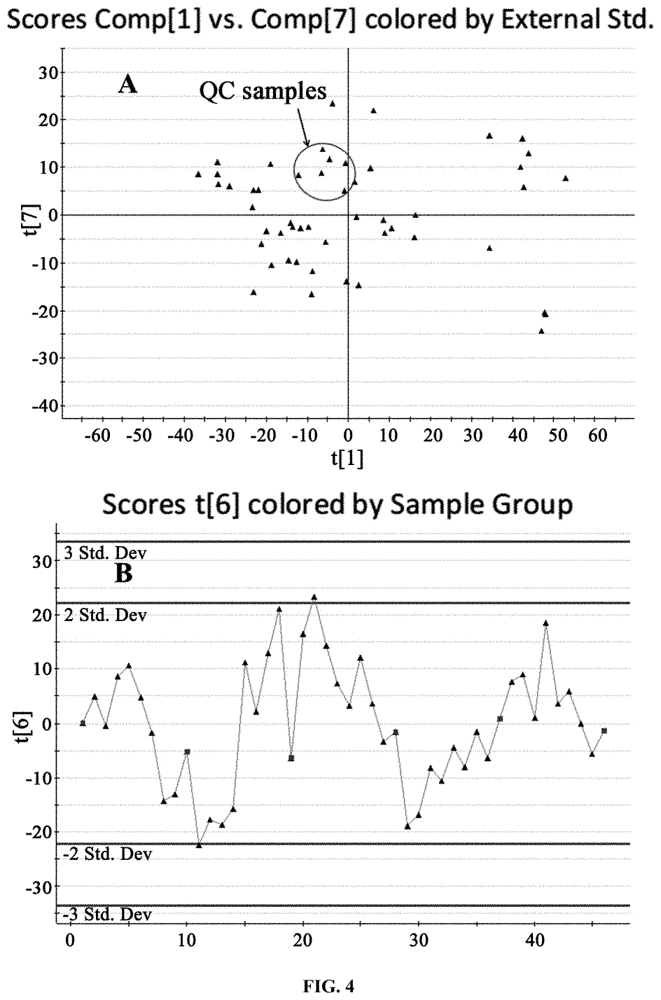

In addition, 10 serum (or urine) samples were randomly selected from each group and mixed together as a QC sample. Since the QC sample contained most of the information of all samples,the QC sample was used to optimize UPLC-QTOF/MS conditions. Before the analysis of samples, the QC sample was injected six times in succession in order to adjust or balance the instrumental system, and during the sample analysis, after each 10 samples was analyzed the QC sample was injected twice to further monitor the analysis stability. After calibration of the instrumental system every day, the QC sample was analyzed firstly to test the stability of the instrument to ensure the consistence of the results. The stability of the QC sample and the relative standard deviation (RSD %) of ion intensitywere illustrated in FIG. 4 and Table 4, respectively. The trend chart showed the change of t[1] in all observations (FIG. 4B). The m/z values of the selected 10 molecules were extracted for methodological validation. The repeatability of the method was evaluated using 6 replications of the QC sample,and the results demonstrated that the method had good repeatability and stability.

TABLE 1
Coefficient of variation of ion intensity of selected ions present in QC samples

| T_{R}_{_{—}}m/z pairs | QC1 | QC2 | QC3 | QC4 | QC5 | QC6 | RSD % |
|---|---|---|---|---|---|---|---|
| 6.94_505.1976 | 16.7211 | 16.8472 | 17.631 | 17.7943 | 15.6255 | 16.4298 | 4.75 |
| 1.63_203.0410 | 10.1133 | 11.2718 | 10.0421 | 10.1354 | 10.4537 | 10.6013 | 4.44 |
| 10.78_284.1824 | 19.5457 | 18.2651 | 19.7107 | 20.9297 | 19.7134 | 19.7996 | 4.31 |
| 10.08_301.1500 | 30.2126 | 28.5028 | 28.2719 | 30.4450 | 29.6215 | 29.0207 | 3.05 |
| 4.52_447.0338 | 3.3463 | 3.5587 | 3.5676 | 3.5204 | 3.1506 | 3.6088 | 5.10 |
| 11.07_259.1846 | 15.0814 | 13.8189 | 14.5798 | 13.8037 | 13.7444 | 14.3567 | 3.78 |
| 7.21_588.2010 | 49.3268 | 47.8549 | 49.4483 | 50.5330 | 49.9091 | 50.2714 | 1.92 |
| 8.40_321.1733 | 5.5704 | 5.6888 | 5.7566 | 5.2010 | 5.0758 | 5.1962 | 5.38 |
| 8.29_556.2156 | 111.0313 | 108.292 | 113.2563 | 112.2356 | 113.4874 | 104.8254 | 3.05 |
| 7.90_482.2067 | 41.4617 | 39.9505 | 36.4553 | 38.8811 | 37.6913 | 38.3039 | 4.52 |

2.3.4 Metabonomic Data Processing and Analysis

The obtained original mass spectrometry data was processed using Masslynx v4.1 software. The main parameters included: retention time range: 0-14 minutes; m/z range: 100-1000; mass tolerance range: 0.01Da; peak intensity threshold: 50; mass window: 0.05Da; retention time window: 0.2 minutes; and automatic detection of 5% peak height and noise. The intensity of each ion was normalized relative to the total ion count to produce a data list consisting of retention time, m/z value and standardized peak area. The data were imported into EZinfo 2.0 for supervised partial least squares discriminant analysis (PLS-DA) and orthogonal partial least squares discriminant analysis (OPLS-DA). The separation of various metabolites between the control group and the model group was shown in the OPLS-DA plot. Potential biomarkers were extracted from the S-plot constructed after OPLS-DA analysis, and each point in the S-plot represented the information of the corresponding variable. The variable importance in the projection (VIP) was measured by the magnitude of the VIP value. The variables were screened according to the VIP value, and the variable with VIP>1 may be selected as a potential biomarker and the molecular formula of the variable of significant difference between the model group and the control group was further identified. Cross validation parameters R²Y and Q² were used to describe the PLS-DA score plot, which represented the total explanatory variables of the X matrix and the predictability of the model, respectively. When a cumulative value of R²Y and Q² was greater than 0.8, the model may be considered to be reliable. The relative distances between other groups and the control group in the PLS-DA score plot were calculated using the average values of all samples (X and Y axes) of the control group as reference points. This quantitative value can be used as an evaluation index of pharmacodynamics and metabonomics to solve many problems in lacking of accurate and quantitative evaluation methods for pharmacodynamics. SPSS 21.0 software was used for statistical analysis of the relevant data. T-test analysis was performed among groups to verify the significance of the difference of potential biomarkers in different groups. The data were expressed as mean±SD,and the data were considered to have a statistical significance with P<0.05.

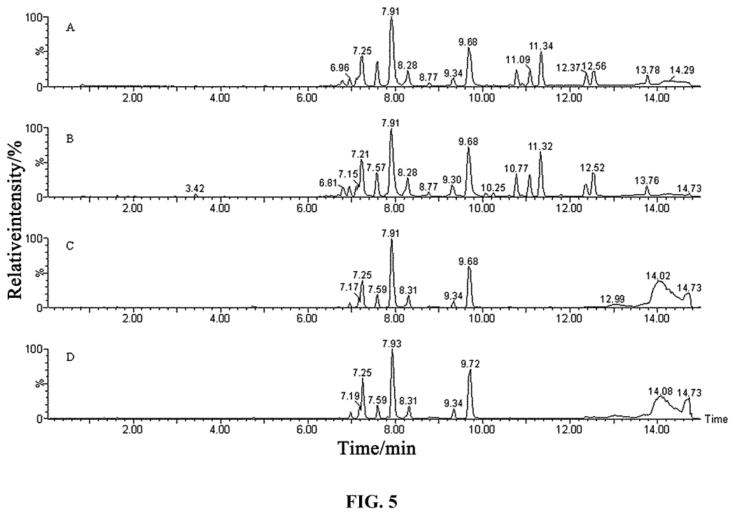

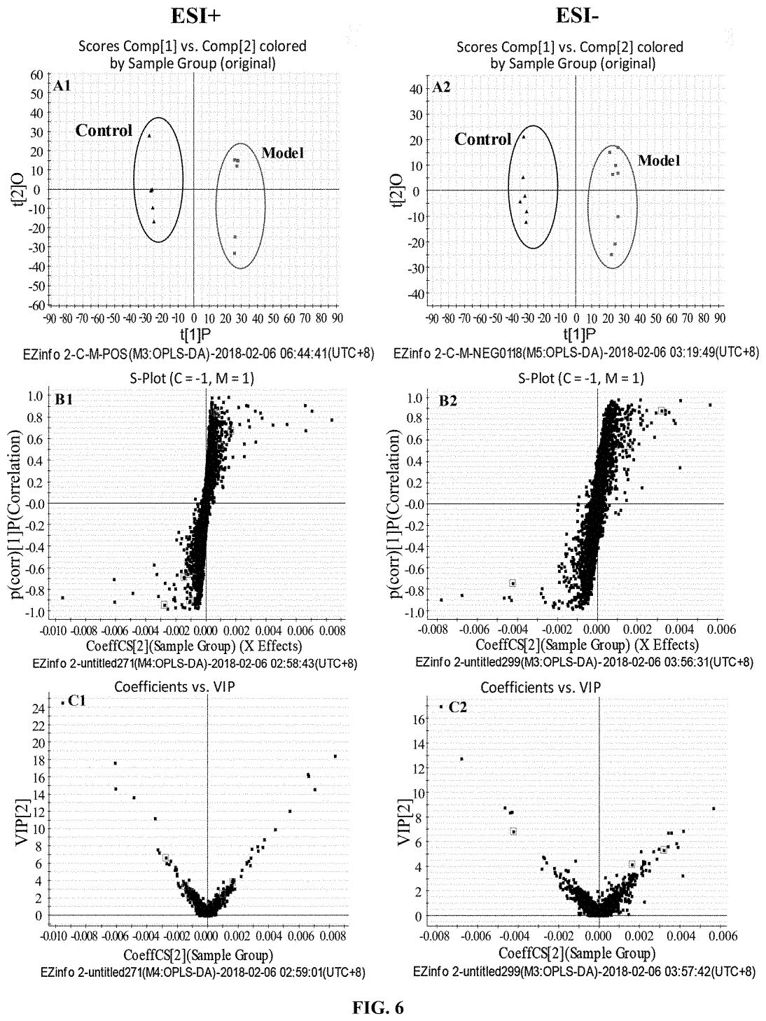

After pretreatment, all collected serum samples were used for separation and original data collection using UPLC-QTOF/MS,and data collection and analysis were carried out using Masslynx4.1 data management software. The samples were scanned under positive and negative ion modes to obtain a base peak total ion flow graph (BPI). FIG. 5 showed typical BPI total ion flow graphs of the mice of the model and the control group under positive and negative ion modes. In order to investigate the overall changes of metabolites in db/db mice, OPLS-DA was performed to analyze the data. As shown in the OPLS-DA score plots (FIG. 6) of the control group and the model group, the two groups of mice can both be significantly discriminated under positive and negative ion modes, indicating that compared to the control group, in-vivo abnormal metabolism was developed in db/db mice of the model group. In the S-plot (FIG. 6), a farther distance between the metabolite and the main ion cluster indicated a greater contribution to the separation of sample groups. FIG. 6 listed R²Y and Q² of serum samples under positive and negative modes in the OPLS-DA plot, indicating that the PLS-DA model may be used for subsequent analysis.

2.3.5 Analysis and Identification of Potential Biomarkers in Mouse Serum

Potential biomarkers were searched and identified in HMDB (http://www.hmdb.ca/) and KEGG (http://www.genome.jp/kegg/) databases based on their tandem mass spectrometry data, and a total of 13 potential biomarkers was identified. The information of mass spectrometry data and the variation trend in serum of mice in the control and model groups were shown in Table 2. The relative contents of these 13 potential biomarkers were analyzed using t-test. Significant differences were observed between the control group and the model group (P<0.05), and see Table 3 for details.

TABLE 2
Potential biomarkers identified in serum of mice

| | | | Molecular | | | | | Metabolic |
|---|---|---|---|---|---|---|---|---|
| No. | T_{R}/min | m/z | formula | Biomarkers | VIP | Trend | HMDB | pathway |
| Sm1 | 8.31 | 482.3617 | C₃₀H₅₉NO₃ | Ceramide(d18:1/12:0) | 7.00 | ↓ | 04947 | Sphingolipid |
| | | | | | | | | metabolism |
| Sm2 | 9.93 | 550.3868 | C₂₈H₅₆NO₇P | PC(18:1(9Z) e/2:0) | 3.68 | ↓ | 11148 | Ether lipid |
| | | | | | | | | metabolism |
| Sm3 | 10.14 | 510.3943 | C₂₆H₅₆NO₆P | LysoPC(O-18:0) | 2.69 | ↓ | 11149 | Ether lipid |
| | | | | | | | | metabolism |
| Sm4 | 10.78 | 351.2310 | C₂₀H₃₀O₅ | PGH3 | 1.18 | ↑ | 13040 | Arachidonic acid |
| | | | | | | | | metabolism |
| Sm5 | 3.44 | 462.2683 | C₂₃H₂₇NO₉ | Morphine-3-glucuronide | 2.47 | ↑ | 41936 | Drug metabolism- |
| | | | | | | | | cytochrome |
| | | | | | | | | P450 |
| Sm6 | 1.88 | 172.9586 | C₆H₆O₆ | cis-Aconitic acid | 1.56 | ↑ | 00072 | Glyoxylate and |
| | | | | | | | | dicarboxylate |
| | | | | | | | | metabolism |
| Sm7 | 7.84 | 313.2755 | C₂₀H₄₀O₂ | Arachidic acid | 1.65 | ↓ | 02212 | Biosynthesis of |
| | | | | | | | | unsaturated fatty |
| | | | | | | | | acids |
| Sm8 | 9.71 | 524.9743 | C₁₀H₁₅N₄O₁₅P₃ | Xanthosine 5-triphosphate | 2.35 | ↓ | 00293 | Purine |
| | | | | | | | | metabolism |
| Sm9 | 3.74 | 347.2236 | C₂₁H₃₀O₄ | Cortexolone | 1.68 | ↑ | 00015 | Steroid hormone |
| | | | | | | | | biosynthesis |
| Sm10 | 8.00 | 522.3589 | C₂₆H₅₂NO₇P | LysoPC(18:1(9Z)) | 3.82 | ↑ | 02815 | Glycerophospholipid |
| | | | | | | | | metabolism |
| Sm11 | 12.36 | 255.1801 | C₁₆H₃₂O₂ | Palmitic acid | 7.15 | ↓ | 00220 | Fatty acid |
| | | | | | | | | metabolism |
| Sm12 | 11.08 | 303.1694 | C₁₉H₂₈O₃ | 16a-Hydroxydehydroisoandrosterone | 4.96 | ↑ | 00352 | Steroid hormone |
| | | | | | | | | biosynthesis |
| Sm13 | 10.25 | 277.1615 | C₁₄H₁₈N₂O₄ | N1-(alpha-D-ribosyl)-5,6- | 5.87 | ↑ | 11112 | Riboflavin |
| | | | | dimethyl-benzimidazole | | | | metabolism |

Note:
the trend was obtained based on model group vs. control group: ↑ content increase, and ↓ content decrease.

TABLE 3
Relative content of 13 endogenous metabolites in control group
and model group (mean ± SD, n = 6)

| No. | Control | Model | P |
|---|---|---|---|
| Sm1 | 8.3 ± 0.20 | 19.84 ± 1.07 | 0.00014 |
| Sm2 | 9.53 ± 1.03 | 5.03 ± 0.46 | 1.2E−06 |
| Sm3 | 3.03 ± 0.54 | 1.62 ± 0.04 | 0.00027 |
| Sm4 | 0.68 ± 0.09 | 1.06 ± 0.24 | 0.00401 |
| Sm5 | 1.13 ± 0.57 | 2.52 ± 0.77 | 0.00397 |
| Sm6 | 1.87 ± 0.19 | 4.56 ± 1.10 | 0.00109 |
| Sm7 | 2.62 ± 0.32 | 1.93 ± 0.36 | 0.00291 |
| Sm8 | 3.5 ± 0.56 | 1.84 ± 0.51 | 0.00464 |
| Sm9 | 0.56 ± 0.18 | 1.18 ± 0.22 | 0.00009 |
| Sm10 | 17.68 ± 2.01 | 22.02 ± 3.12 | 0.01181 |
| Sm11 | 124.21 ± 13.68 | 88.34 ± 8.62 | 0.00049 |
| Sm12 | 128.97 ± 9.63 | 157.09 ± 10.36 | 0.00256 |
| Sm13 | 9.87 ± 2.50 | 30.78 ± 5.25 | 0.00004 |

2.3.6 Evaluation of Intervention Effect of Verbascoside on db/db Mice

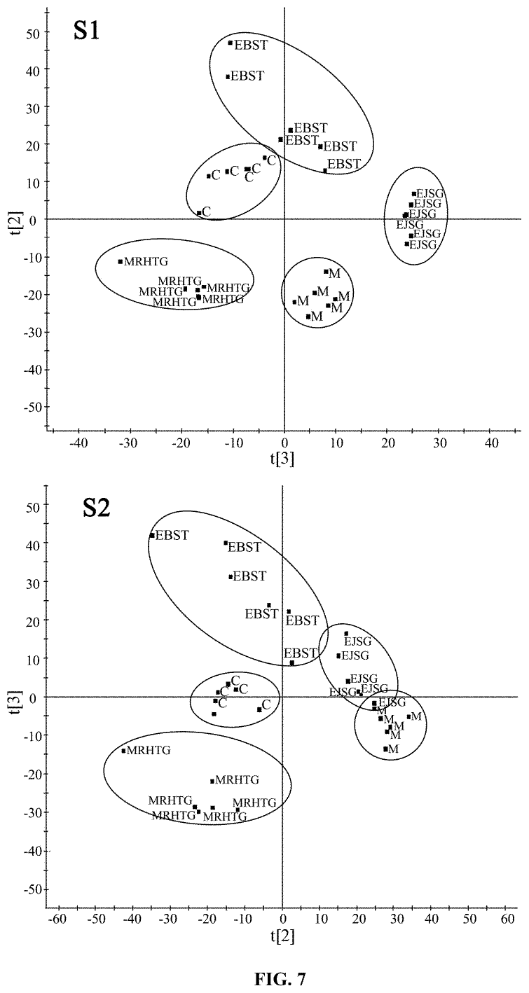

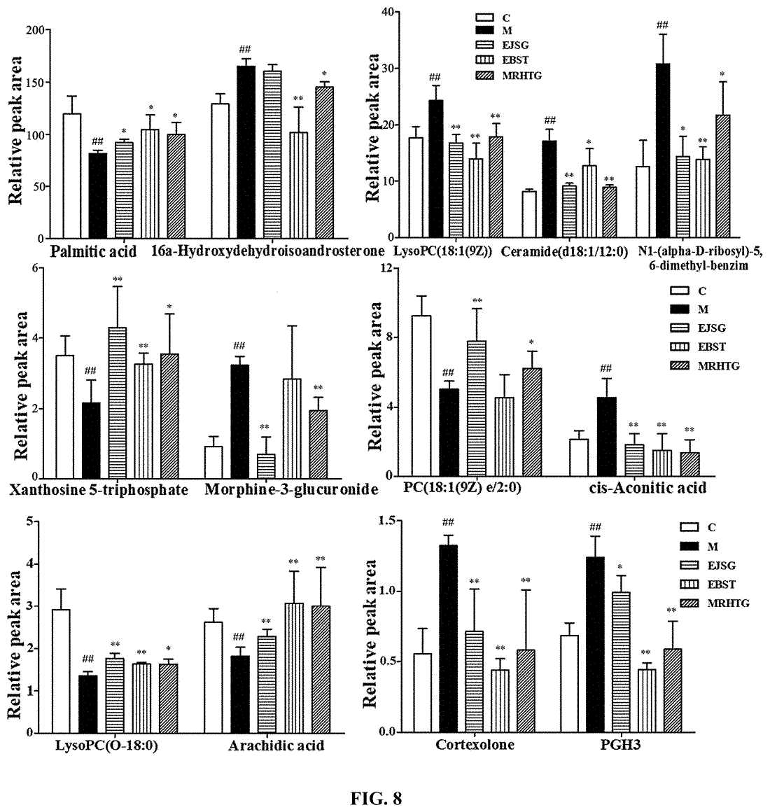

A PLS-DA model was established for analysis to obtain the changes of mice among the control group, model group and administration group (FIG. 7) so as to investigate the intervention effect of verbascoside on db/db mice and its action mechanism. Relative contents of 13 endogenous metabolites in serum of mice in the control group, model group and administration group were analyzed using t-test. The results obtained using UPLC-QTOF/MS analysis showed that there were significant differences between the control group and the model group in the content of endogenous metabolites in the serums. Compared to the model group, the levels of the 13 endogenous metabolites in the serums of the administration group showed a trend to return to those in the serums of the control group to different extents (Table 4). The detailed information was shown in FIG. 8.

TABLE 4
Relative distance between administration group and control group
in PLS-DA score plot (mean ± SD, n = 6)

| | | |
|---|---|---|
| ESI | + | − |

| | | | |
|---|---|---|---|
| C | X-Axis | −10.21 | −14.4 |
| | Y-Axis | 11.53 | −0.39 |

| | | |
|---|---|---|
| M | 36.62 ± 3.28 | 43.46 ± 3.15 |
| EJSG | 36.57 ± 1.53 | 34.94 ± 2.74** |
| EBST | 21.56 ± 8.01*** | 32.16 ± 10* |
| MRHTG | 31.62 ± 1.07*** | 28.59 ± 3.33*** |

Note:
the results were obtained by comparing with model group, *P < 0.05; **P < 0.01; ***P < 0.001.

2.4 Human Renal Tubular Epithelial (HK-2) Cells

2.4.1 Cell Culture and Grouping Administration

Human renal tubular epithelial cells (HK-2) were provided by Nanjing KeyGEN Biotechnology Development Co., Ltd. The complete medium was a low-sugar DMEM with 10% FBS and 1% penicillin-streptomycin solution. The cells were culturedat 37° C. under 5% CO₂ and saturatedhumidity in anincubator. Cells were spread in a 96-well plate and cultured with a 5 mmol/Llow-sugar DMEM for 24 hours. Subsequently, the cells were divided into five groups including control group (C), without intervention factors; osmotic pressure control group (DMEM containing 24.5 mmol/L mannitol, GLC); model group (M), with cells cultured in a high-sugar DMEM (30 mmol/L) solution; and administration group, with cells cultured in DMEM high-sugar (30 mmol/L) solutions respectively containing 5, 10, 25, 50, 100 μmol/L of irbesartan (EBST) and respectively containing 5, 10, 25, 50, 100 μmol/L of verbascoside (MRHTG). The number of cells in each group was detected using a cell counting method after 48 hours of the incubation.

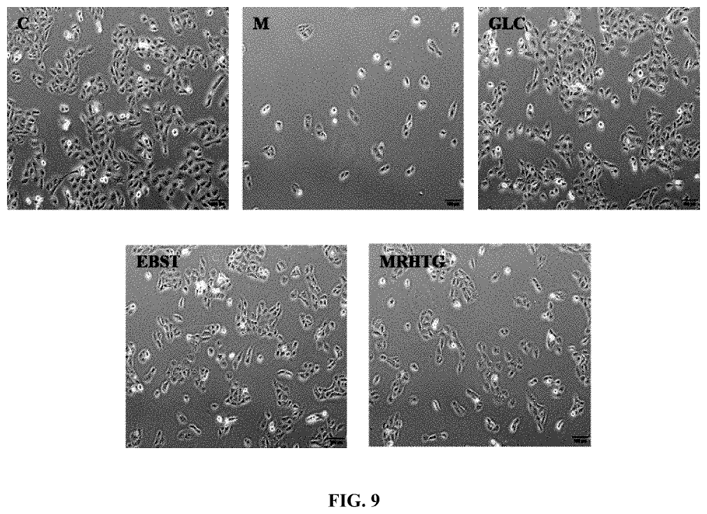

After HK-2 cells in the control group, model group, 50 μmol/L EBST and 50 μmol/LMRHTG administration groups were cultured for 48 hours, the cell morphologies were shown in FIG. 9. The cells in the control group were paving stone-shaped, and the cells in the model group with high-glucose inducement were sparse in a spindle or fibrous shape. The cell morphologies of 50 μmol/LEBST and 50 μmol/LMRHTG administration groups were close to those of the control group.

2.4.2 Influence of Verbascoside on Proliferation of HK-2 Cells

As shown in Table 5, high glucose significantly inhibited the proliferation of HK-2 cells, while the intervention of verbascoside (5, 10, 25, 50, 100 μmol/L) could promote the proliferation of HK-2 cells under a high glucose condition, and the promotion of the proliferation was dose-dependent.

TABLE 5
Influence of verbascoside on proliferation of
high-glucose induced HK-2 cells

| | | | inhibition |
|---|---|---|---|
| Groups | Concentration μmol · L⁻¹ | OD | ratio % |
| Control group | | 0.632 ± 0.016 | — |
| Model group | | 0.345 ± 0.007 | — |
| Mannitol group | | 0.604 ± 0.014 | 90.07 |
| Irbesartan group | 50 | 0.576 ± 0.02 | 80.57 |
| | 5 | 0.418 ± 0.008 | 25.52 |
| | 10 | 0.457 ± 0.014 | 39.02 |
| MRHTG | 25 | 0.501 ± 0.015 | 54.36 |
| | 50 | 0.557 ± 0.012 | 73.95 |
| | 100 | 0.559 ± 0.01 | 74.65 |

2.4.3 Influence of Verbascoside on Protein Expression of NOX1, NOX2, NOX4, α-SMA, E-cadherin, NF-κB p65, TGF-β1, Smad2, Smad3, Smad4 and Smad7 in HK-2 Cells

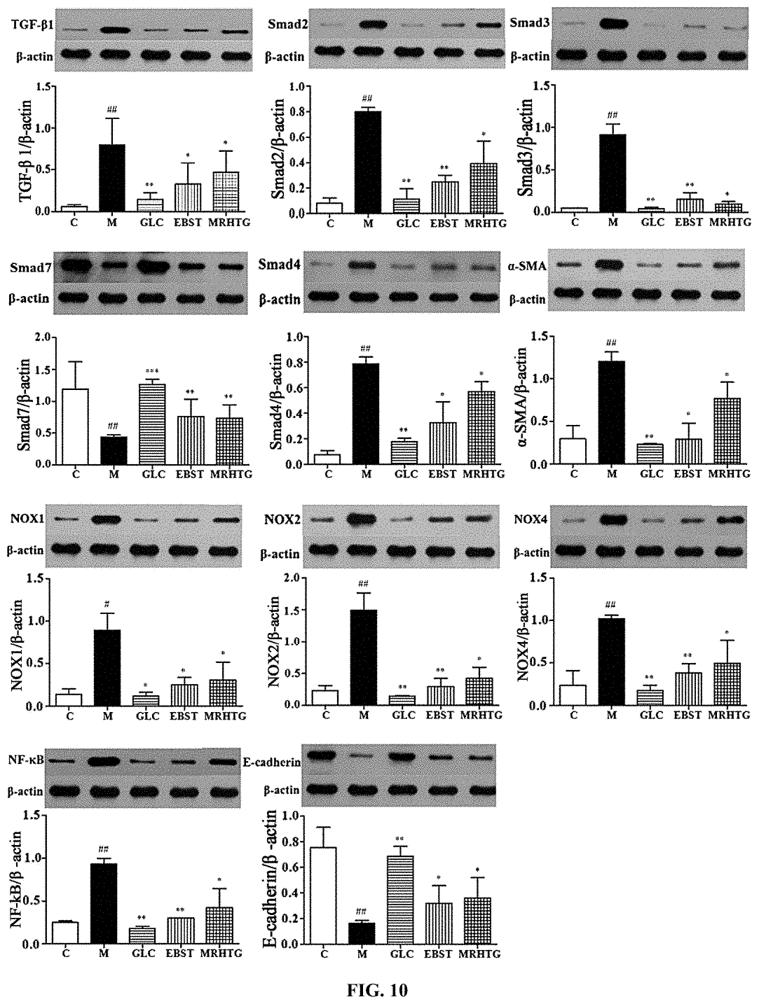

As shown in FIG. 10, compared to the control group, the protein expression levels of NOX1, NOX2, NOX4, α-SMA, NF-κB p65, TGF-β1, Smad2, Smad3 and Smad4 in the HK-2 cells with high-glucose induced injuries were increased significantly (P<0.05), while the protein expression levels of E-cadherin and Smad7 were decreased significantly (P<0.05). After intervention with 50 μmol/L of verbascoside, the expressions of NOX1, NOX2, NOX4, α-SMA, E-cadherin, NF-κB p65, NF-κ, Smad2, Smad3, Smad4 and Smad7 all returned to normal.

2.4.4 Influence of Verbascoside on mRNA Expression of NOX1, NOX2, NOX4, α-SMA, E-cadherin, NF-κB p65, TGF-β1, Smad2, Smad3, Smad4 and Smad7 in HK-2 Cells

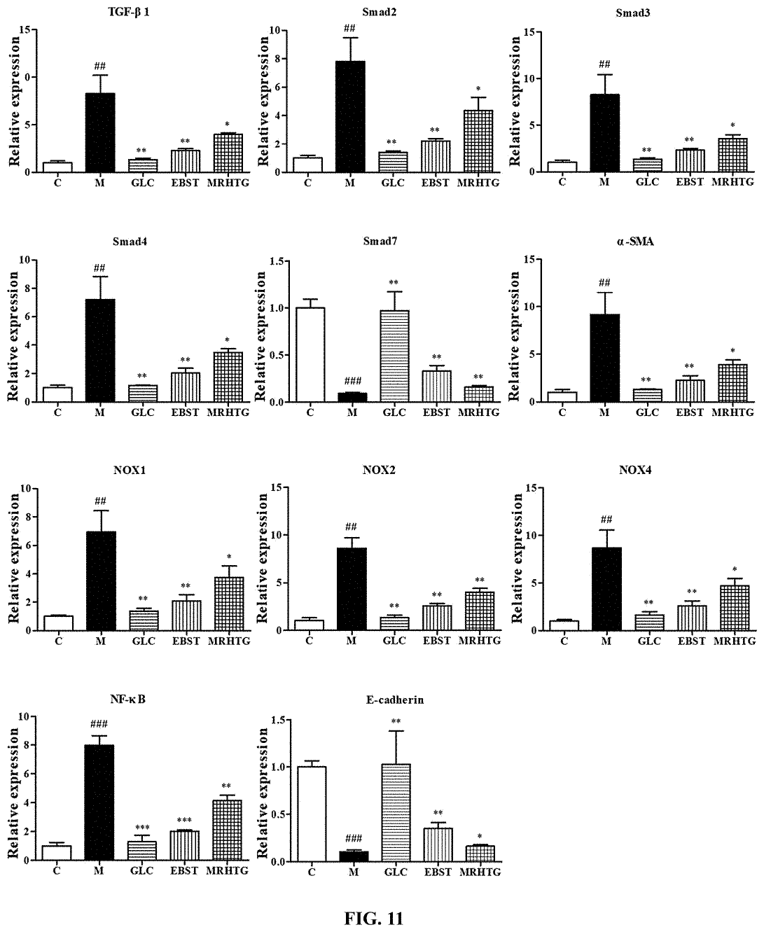

As shown in FIG. 11, compared to the control group, the mRNA expressions of NOX1, NOX2, NOX4, α-SMA, E-cadherin, NF-κB p65, TGF-β1, Smad2, Smad3, Smad4 and Smad7 were consistent with their protein expressions in HK-2 cells with high glucose-induced injuries. After being intervened with 50μmol/L of verbascoside, the mRNA expressions of NOX1, NOX2, NOX4, α-SMA, E-cadherin, NF-κB p65, TGF-β1, Smad2, Smad3, Smad4 and Smad7 all returned to normal.

2.4.5 Influence of Verbascoside on Secretion of MCP-1, IL-1β, TNF-α a and IL-6 in Supernatant of HK-2 Cells

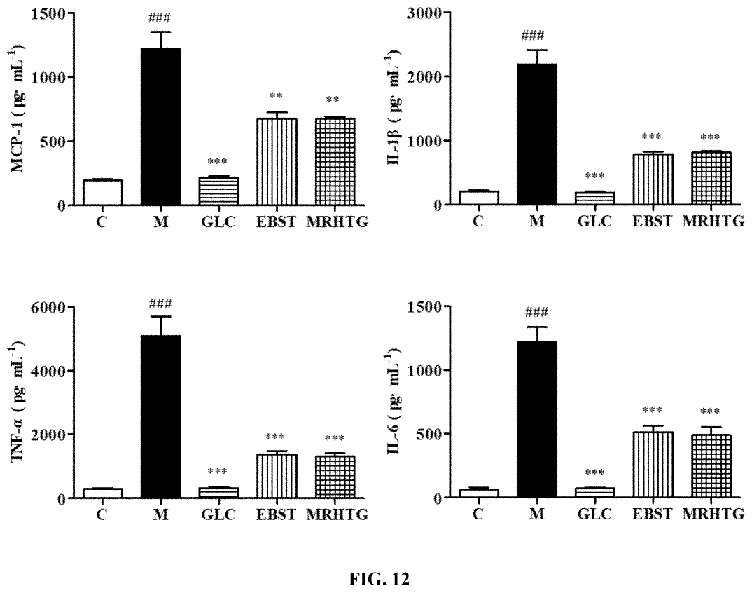

As shown in FIG. 12, compared to the control group, the expression levels of MCP-1, IL-1β, TNF-α and IL-6 in the supernatant of the HK-2 cells with high glucose-induced injuries were increased significantly (P<0.05). After being intervened with 50 μmol/L of verbascoside, the expression levels of MCP-1, IL-1β, TNF-α and IL-6 returned to normal.

3. Discussion

Db/db diabetic mouse model was internationally recognized as a diabetic animal model. Investigations have shown that the occurrence mechanism of diabetic nephropathy (DN) in db/db mice was similar to that in humans, so that the db/db mice were often used to study the pathogenesis of DN. It was found that the male db/db mice of this strain developed hyperglycemia (FBG≥16 mmol/L) at 6-10 weeks, microalbuminuria at 10-12 weeks, and obvious renal function injuries at 15-18 weeks. Herein, male db/db mice, aged 6-8 weeks,were selected, and fed adaptively for 3 weeks followed by an administration for 6 weeks. After the administration, obvious renal function injuries have been observed in mice in the model group. Therefore, the db/db mouse model in this experiment may be used to study the pathogenesis of DN and therapeutic effects thereof.

TGF-β1 and its signal transduction proteins Smad3 and Smad4 played a key role in renal fibrosis. It has been found that Smad3 may promote renal fibrosis by directly binding to the promoter region of collagen, inhibit the degradation of extracellular matrix (ECM) by inducing matrix metalloproteinase inhibitor-1, and simultaneously reduces the activity of matrix metalloproteinase-1 in fibroblasts. It has also been proved that the absence of Smad4 in mesangial cell may inhibit the deposition of ECM induced by TGF-β1, and the specific absence of Smad4 in renal tubular epithelial cells may inhibit the activity of Smad3 response promoter to attenuate renal fibrosis induced by unilateral ureteral obstruction, and reduce the binding of Smad3 to target genes, without depending on its phosphorylation and nuclear translocation. Studies have shown that α-smooth muscle actin (α-SMA) was a marker protein of myofibroblasts, and the activation of Smad3 may promote the expression of α-SMA and renal fibrosis.

The results of biochemical analysis showed that, compared to db/db model group, the levels of serum creatinine, urea nitrogen and microalbuminuria were significantly improved in the treatment group, and the levels of blood lipid (total cholesterol and triglyceride) and blood glucose (fasting blood glucose and insulin levels) were also improved to different extents in the treatment group. In addition, the levels of the liver indexes (glutamic-oxalacetic transaminase and glutamic-pyruvic transaminase) were increased significantly in the model group, indicating the development of liver injury caused by high glucose, which may be improved with TLR. Western blotting results showed that the expressions of TGF-β1 and its signal transduction proteins Smad3 and Smad4 and α-SMA in kidney tissues were decreased significantly, especially after administration of TFR extract. In conclusion, TLR may reduce the contents of microalbuminuria, serum creatinine and urea nitrogen, improve fasting blood glucose, insulin level and dyslipidemia, reduce the expressions of TGF-β1/Smad signal pathway and α-SMA protein and improve renal interstitial fibrosis, thus playing a role in improving the kidney injury.

The progression of diabetic nephropathy was considered to be an irreversible process and could result in end-stage renal failure characterized by extensive renal fibrosis. Renal fibrosis was characterized by the activation of a large number of interstitial myofibroblasts, which was thought to play a central role in the pathogenesis of renal interstitial fibrosis. Myofibroblast activation played a key role in the development of chronic kidney diseases. New evidence showed that myofibroblasts may be derived from renal tubular epithelial cells through epithelial mesenchymal transformation (EMT). Among the identified factors, it has been shown that the main pathogenic driver inducing EMT or binding with other mediators in renal tubular epithelial cells was the profibrotic cytokine TGF-β1, which acted as a growth regulator to inhibit the growth of most types of cells, including epithelial cells, but stimulate the growth of some mesenchymal cells. Studies have shown that TGF-β1-induced renal fibrosis was mainly achieved with TGF-β1 and Smad-related proteins (Smad2, Smad3, Smad4 and Smad 7). The absence of E-cadherin expression was a marker of EMT. The destruction of E-cadherin mediated by matrix metalloproteinase directly led to EMT in renal tubular epithelial cells through Slug. In this section of the study, we found that the protein and mRNA expressions of TGF-β1, Smad2, Smad3 and Smad4 in high glucose-induced HK-2 cells were up-regulated, while the protein and mRNA expressions of inhibitory Smad? and E-cadherin were down-regulated.

It was reported that the increase of intracellular reactive oxygen species (ROS) level played an important role in the development of tubulointerstitial fibrosis and subsequent renal fibrosis. High glucose-induced ROS was mainly formed with the action of NADPH oxidase in renal epithelial cells. The prototype NADPH oxidase firstly identified in phagocytes consisted of two membrane-bound subunits, and NOX1, NOX2 and NOX4 were detected in renal cells, and NOX4 was the most abundant among them. In addition, the increased ROS was associated with renal fibrosis, and promoted the production of collagen, fibronectin and α-SMA. The increased ROS also played a key role in inflammation through the NF-κB pathway. In this study, we found that the protein and mRNA expressions of NOX1, NOX2, NOX4, α-SMA, and NF-κB p65 in high glucose-induced HK-2 cells were increased, and the expressions of NF-κB downstream inflammatory cytokines MCP-1, IL-1β, TNF-α and IL-6 in the cell supernatant were increased significantly (P<0.05).

The results of in vitro studies revealed that TLR, verbascoside and catalpol had an effect on inhibiting the proliferation of HK-2 and the formation of EMT, which were consistent with the results of the study in the first section of this chapter. The results of invitro researches further demonstrated that TLR, verbascoside and catalpol may exert their renal protection effect by regulating oxidative stress response and TGF-62 /smad signal pathway.

In other embodiments, the above verbascoside may be prepared using the following method, which includes the following steps with leaves of Rehmannia used as a raw material.

(1) Dried leaves of Rehmannia were pulverized properly and then immersed in water or an ethanol solution of a low concentration (5%-50%) used as an extracting solvent to produce a blend.The blend was refluxed, filtered and concentrated. The concentrated solution was added with a high-concentration ethanol to an ethanol concentration of 80% for precipitation. The ethanol precipitation was performed by standing overnight. Then the reaction was filtered to obtain a supernatant. The supernatant was concentrated to obtain a crude extract of verbascoside.

(2) The crude extract of verbascoside was separated and purified with AB-8 macroporous adsorption resin in gradient elution to collect a verbascoside-containing eluent. The verbascoside-containing eluent was concentrated to obtain a verbascoside-enriched part with a concentration greater than 60%. (3) The verbascoside-enriched part was dissolved in water to produce a solution. The solution was subjected to an adsorption with macroporous adsorption resin or polyamide resin and then eluted with ethanol in gradient manner. The eluent rich in verbascoside was collected and concentrated followed by spray drying to a refined verbascoside with content greater than 95%.

The above method for preparing verbascoside involved an easy operation, a low cost and a product of high purity, so that it was more favorable for industrial production and the application of verbascoside in the above aspects for treating and preventing diabetic nephropathy.

Obviously, the above embodiments of the present invention are merely used to clearly describe the present invention, but are not intended to limit the embodiments of the present invention. Other forms of variations or modifications may be made by those skilled in the art on the basis of the above description. There is no need and no way to exhaust all of the embodiments. These obvious variations or modifications made without departing from the spirits of the present invention should still fall within the scope of the present invention.

What is claimed is:

1. An application of verbascoside in the preparation of drugs or healthcare products for preventing or treating a diabetic-associated kidney injury in any one of:
1) reducing TGF-β1/Smad signal pathway and α-SMA protein expression;
2) inhibiting formation of EMT;
3) reducing level of intracellular reactive oxygen species;
4) reducing protein expression quantity of one or more of NOX1, NOX2, NoX4, α-SMA, NF-κB p65, TGF β1, Smad2, Smad3 and Smad4;
5) reducing expression quantity of one or more of MCP-1, IL-1/β, TNF-α and IL-6; and
6) increasing protein expression quantity of E-cadherin and/or Smad7.

2. The application according to claim 1, wherein the diabetic-associated kidney injury is a type II diabetic kidney injury.

3. The application according to claim 1, wherein the diabetic-associated kidney injury comprises at least one of tubulointerstitial fibrosis, glomerular hyperfiltration, renal hypertrophy and forepart glomerular sclerosis.

4. The application according to claim 1, wherein the application is an application of verbascoside in the preparation of drugs or healthcare products for preventing or treating a livery injury caused by a diabetic kidney injury.

5. The application according to claim 1, wherein the drugs or healthcare products is administered by means of oral administration, parenteral administration, mucosal administration or transdermal administration.

6. The application according to claim 1, wherein the drugs or healthcare products comprise tablets, capsules, granules, oral liquid, patches and gels.

7. A method for preventing or treating a diabetic-associated kidney injury, comprising:
using verbascoside to
reduce TGF-β1/Smad signal pathway and α-SMA protein expression, and/or inhibit formation of EMT, and/or
reduce level of reactive oxygen species in cells, and/or
reduce protein expression quantity of one or more of NOX1, NOX2, NoX4, α-SMA, NF-κB p65, TGF-β1, Smad2, Smad3 and Smad4, and/or
reduce expression quantity of one or more of MCP-1, IL-1β, TNF-α and IL-6, and/or
increase protein expression quantity of E-cadherin and/or Smad7.

8. The method according to claim 7, wherein the diabetic-associated kidney injury is a type II diabetic-associated kidney injury and comprises at least one of tubulointerstitial fibrosis, glomerular hyperfiltration, renal hypertrophy and forepart glomerular sclerosis.
